(19) United States
(12) Patent Application Publication
(10) Pub. No.: US 2007/0009562 A1
(43) Pub. Date: Jan. 11, 2007

(54) Spherical optical structure

(76) Inventor: Fumiyoshi Ikkai, Kawasaki (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/472,283

(22) Filed: Jun. 22, 2006

Related U.S. Application Data
(60) Provisional application No. 60/752,403, filed on Dec. 22, 2005.

(30) Foreign Application Priority Data
Jun. 22, 2005 (JP) JP2005-182696
Nov. 25, 2005 (JP) JP2005-340979

(51) Int. Cl. A61K 8/81

(52) U.S. Cl. 424/401; 977/926; 424/70.16

(57) ABSTRACT

Disclosed herein is a spherical optical structure having a particle diameter less than or equal to 500 μm, comprising a core substance and a coating substance coating the core substance, wherein the particle diameter and/or the thickness of the coating substance of the spherical optical structure are controlled to be in a predetermined range, according to the refractive index of an external medium in contact with the outer surface of the spherical optical structure and the refractive index of the coating substance, so as to display a structural color in the visible light range. Also disclosed herein is a cosmetic coloring material comprising the spherical optical structure and a cosmetic composition comprising the coloring material.

This application claims benefit of U.S. Provisional Application No. 60/752,403, filed Dec. 22, 2005, the contents of which are incorporated herein by reference. This application also claims benefit of priority under 35 U.S.C. § 119 to Japanese Patent Application No. 2005-182696, filed Jun. 22, 2005, and Japanese Patent Application No. 2005-340979, filed Nov. 25, 2005, the contents of which are also incorporated herein by reference.

Disclosed herein is a spherical optical structure capable of displaying a structural color. Also disclosed herein is a cosmetic coloring material comprising the spherical optical structure and a cosmetic composition comprising the coloring material.

In the field of cosmetic products, pigments, colorants, dyes, and other such substances are widely used as coloring materials for coloring cosmetic ingredients. Coloring may be carried out by mixing these coloring materials into a cosmetic ingredient composition, so that the cosmetic ingredient being employed becomes affixed with pigment and/or colorant, and/or dyed with a dye. In the case of such conventional coloring materials as these pigments, colorants, and/or dyes, differences may occur in the wavelength characteristics of the light reflected from the surface of the coloring material due to differences in the wavelength-dependency of absorbance, i.e., due to differences in the absorption spectrum characteristics, at the surface of the coloring material. As a result, an observer may be cognizant of coloring differences.

In the case of coloring materials such as those described above, the mixing of a plurality of coloring materials has been attempted in order to obtain variety in coloring. In addition, in the case of use as a cosmetic ingredient, additives such as inorganic layered composite powders like mica, pearlescent pigments, and liquid-crystal compounds may be mixed into a cosmetic composition for the purpose of adding gloss and/or luster to the surface of the skin, nails, and/or hair.

For example, a color pearling agent, in which a colored powder such as iron oxide, smalt, chromium hydroxide, and/or carmine is mixed with or coated onto titanium mica, which has a pearl luster and an interference color, is an ingredient that may be essential as a lustrous coloring material in a cosmetic product. See Development of Advanced Cosmetics, CMC Publishing Co., Ltd., 297-306 (2000).

Mixing of conventional coloring materials may lead to a subtractive color mixture due to absorption of light by each of the respective coloring materials. As a result, it is not always possible to generate the unique characteristics of each of the coloring materials, and combining of coloring materials may result in a deterioration in color saturation.

Moreover, additives such as inorganic layered composite powders, pearlescent pigments, and liquid-crystal compounds are themselves colorless or white, or have little variety of color, so that they must be used in combination with other coloring materials in order to obtain the desired coloring. In this case, when such additives are combined with a coloring material that has low color saturation and brightness, it may not always be possible to produce the lustrous sensation desired from these additives.

On the other hand, colored powders that are mixed into the aforementioned color pearling agent, which is a conventional combination deemed to have good color saturation, do not always have superior chemical stability. For example, smalt has poor alkali and thermal resistance, and carmine has poor light fastness.

Further, there are many conventional pigments, colorants, and/or dyes that, depending on the quantity employed, are not entirely harmless with respect to the human body, e.g., effect on skin.

Accordingly, the present disclosure employs a principle that differs from coloring methods using absorption of a portion of light as in the case of conventional coloring materials, and aims to provide a spherical optical structure for a non-toxic, chemically stable cosmetic composition that may display a bright structural color regardless of the direction of view. Also disclosed herein is a cosmetic composition comprising such a spherical optical structure.

The production method of microcapsules is known as a method for creating mass amounts of minute spherical particles having a shell structure. Microcapsules comprise a core substance and a coating, and a wide variety of applications for microcapsules has been investigated. For instance, in the fields of cosmetics and pharmaceuticals, microcapsules have been used for improving the stability of the various effective components in a composition, and imparting sustained release properties, for the blocking of odors and tastes originating from the effective components, and other uses. For example, blue coloring materials wherein pigments and/or dyes are enclosed within microcapsules are known (see, e.g., Japanese Patent Publication (Kohyo) No. 2004-526558). However, no attempt has been made to color by means of the microcapsules themselves, without the use of conventional pigments and/or dyes.

The present inventors have discovered that coloring in a desired structural color in the visible light range can be obtained, using a spherical optical structure having a particle diameter of, for example, less than or equal to 500 μm, comprising a core substance and a coating substance coating the core substance, by controlling the particle diameter and/or the thickness of the coating substance of the spherical optical structure, according to the refractive index of the external medium in contact with the outer surface of the spherical optical structure, and the refractive index of the coating substance.

Structural colors differ from pigment colors, which are based on the absorption of light by substances, in that they are colors that are created based upon the microstructure of substances, and this relates to the scattering and interference of light. In cosmetics, inorganic substance particles having a multilayered thin film structure may be used as angle dependent coloring materials for which the color changes depending upon the viewing angle. However, no examples have been reported of a structural color having been obtained by adjusting the particle diameter and/or the thickness of the coating of spherical optical structures in the form of microcapsules.

The spherical optical structure of the present disclosure may develop a structural color according to the particle diameter and/or thickness of the coating material, without using conventional colorants such as pigments and/or dyes. The structural colors obtained may have a unique transparent feel, and by blending these spherical optical structures into various cosmetics, it may become possible to obtain specific hues that are not possible conventionally.

Additionally, for the spherical optical structure of the present disclosure, the toxicity of the core substances (internal phase) may be suppressed by utilizing appropriate coating substance materials, such as polystyrene and the like, so the safety of these structures may be improved.

### BRIEF DESCRIPTION OF THE DRAWINGS

FIG. 1 is a cross sectional geometrical diagram illustrating optical principles of spherical optical structure displaying structural colors of the present disclosure.

FIG. 2 is a schematic diagram showing a cross section of a spherical optical structure displaying red and blue structural colors of the present disclosure.

FIG. 3 is an optical photomicrograph of spherical optical structures obtained according to Example 1 of the present disclosure.

FIG. 4 is a comparison of transmission spectra of spherical optical structures of each color obtained according to Example 1 of the present disclosure.

The core substance contained in the spherical optical structure of the present disclosure may be a material having a refractive index different from that of the coating substance, and also optionally comprising at least one additional component chosen from aqueous or hydro-organic solutions comprising water and/or at least one C₁-C₆ alcohol such as ethanol and/or isopropanol; liquid substances such as organic liquid substances and/or inorganic solvents, and mixtures thereof; and semiliquid materials having a certain degree of viscosity. For example, if the user desires ease of preparation, the core substance may comprise at least one aqueous or hydro-organic solution, for example, an aqueous solution in which gelatin and/or hydrophilic thickening agents are dissolved. In another embodiment, the core substance may be air (the interior being hollow).

The material constituting the coating may be a material conventionally used for the formation of microcapsules, and may be chosen from materials having a refractive index different from that of the medium in which the spherical optical structures of the present disclosure are blended. Examples of suitable coating materials include, but are not limited to, polymers such as vinyl based polymers such as polystyrene; polyalkylenes such as polyethylene; C₁-C₃₂ alkyl poly(meth)acrylate such as methyl poly(meth)acrylate; poly(meth)acrylamide; alkyl polyacetate such as ethyl polyacetate; polycondensation polymers such as polycarbonate, polyurethane, nylon, and polyester; cellulose based polymers such as cellulose acetate, and ethylcellulose; silicone based polymers such as polyalkylsiloxane, and polydimethylsiloxane; organic polymers such as chloride polymers, and fluorine based polymers; and/or inorganic materials such as glass, silica, and titania.

In one embodiment of the present disclosure, the spherical optical structure has a particle diameter less than or equal to 500 μm, and comprises a core substance and a coating substance coating the core substance, wherein a structural color in the visible light range is displayed by controlling the particle diameter and/or the thickness of the coating substance of the spherical optical structure to be in a predetermined range, according to the refractive index of the external medium in contact with the outer surface of the spherical optical structure and the refractive index of the coating substance.

In another embodiment, the spherical optical structure of the present disclosure has a particle diameter less than or equal to 500 μm, and comprises a core substance and a coating substance that coats the core substance, wherein by controlling the particle radius L and/or the thickness of the coating substance D of the spherical optical structure according to the refractive index n₁ of the external medium in contact with the outer surface of the spherical optical structure and the refractive index n₂ of the coating substance, a structural color with a wavelength A in the visible light range may be obtained by substituting a value d satisfying:

$$\begin{matrix}sin \{ 90 - arccos ( \frac{L - d}{L} ) \} = \frac{{n}_{2}}{{n}_{1}} sin [ \{ 90 - arccos ( \frac{L - d}{L} ) \} - arctan [ \frac{D - d}{L sin \{ arccos ( \frac{L - d}{L} ) \}} ] ] \\ into & Equation 1 \\ \frac{λ}{2} ( 2 m + 1 ) ( m = 0 , 1 , 2 , 3 , … ) = \frac{2 L sin \{ arccos ( \frac{L - d}{L} ) \}}{cos [ arctan [ \frac{D - d}{L sin \{ arccos ( \frac{L - d}{L} ) \}} ] ]} - 2 L sin \{ arccos ( \frac{L - d}{L} ) \} & Equation 2\end{matrix}$$

wherein m is an integer greater than or equal to 0.

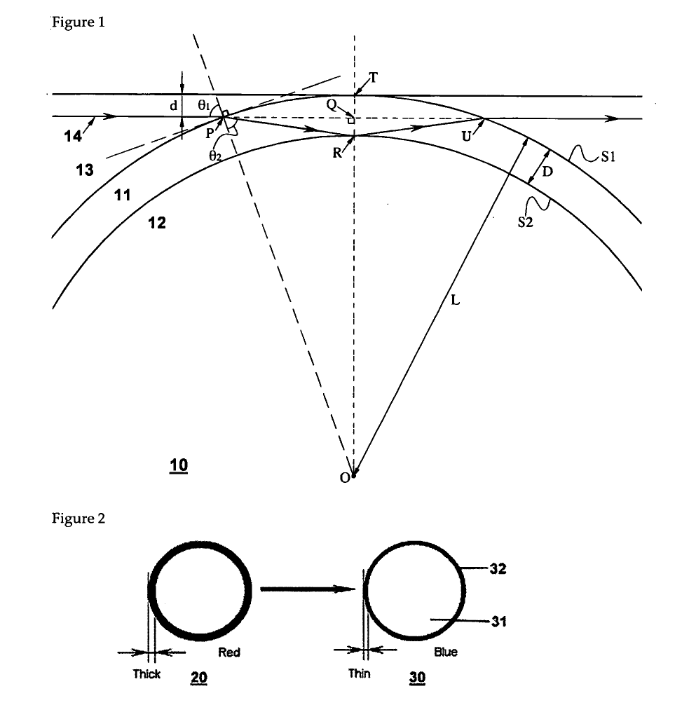

FIG. 1 is a diagram illustrating the difference in the length of the light path, in comparison with directly propagating light, created by the geometrical relationship between the particle radius L of the spherical optical structure of the present disclosure, the thickness D of the coating substance, the coating substance (refractive index n₂), and the external medium in contact with the outer surface of the aforementioned spherical optical structure (refractive index n₁).

A light beam 14 enters through an external medium 13 (refractive index n₁) in contact with the outer surface of a spherical optical structure, is refracted according to Equation 1 derived from Snell's Law (n₁ sin θ₁=n₂ sin θ₂) at point P on the coating outer wall S1 that is the surface of the boundary with the coating 11 (refractive index n₂) of the spherical optical structure 10, and enters the internal portions of the coating 11. The incident light beam is reflected at point R on the coating inner wall S2 of the coating 11, and exits back into the external medium 13 from point U on the coating outer wall S1. At this time, at point U, interference occurs between the light beam 14 that has propagated through the path going through the abovementioned point P, point R, and point U, and light that has propagated through the external medium 13 through a distance corresponding to the straight line between point P and point U. In this case, for a wavelength λ that satisfies the left side of Equation 2 for the light path difference arising between the two light beams (corresponding to the right side of Equation 2), conditions arise wherein the light is strengthened due to constructive interference effects.

Therefore, an optimal spherical optical structure can be obtained for a wavelength λ for which coloring by a structural color is desired, by controlling the thickness D of the coating and/or the particle radius L of the spherical optical structure so as to simulatneously satisfy Equation 1 and Equation 2.

Since the m on the left side of Equation 2 is an integer greater than or equal to 0, and the interference effects are strongest at m=0, the spherical optical structure of the present disclosure may be primarily designed for the condition m=0. However, colors for the conditions of m=1 or above may also be obtained at the same time.

As used herein, the phrase, “controlling the particle diameter and/or the thickness of the coating substance of the spherical optical structure to be in a predetermined range, according to the refractive index of the external medium in contact with the outer surface of the spherical optical structure and the refractive index of the coating substance” means that, for a given combination of the refractive index of the external medium in contact with the outer surface of the spherical optical structure and the refractive index of the substance selected as a coating substance, the thickness of the coating substance is produced so as to be within a range suitable for displaying a desired structural color, for a given particle diameter of the spherical optical structure.

Further, for a given combination of the refractive index of the external medium in contact with the outer surface of the spherical optical structure and the refractive index of the substance selected as a coating substance, a plurality of pairs of combinations of the particle diameter of the spherical optical structure and the coating thickness exists, but, in at least one embodiment, it may be sufficient for the dimensions of the produced spherical optical structures to be within a specific range that results in the development of the desired structural color.

In addition to the exact matching of the coating thickness to the thickness D that satisfies Equation 1 and Equation 2 whereby the wavelength λ of the desired structural color is derived, thicknesses whereby the structural coloring can be developed that roughly match the thickness D are also included. Further, in addition to cases wherein the light path difference created between a light beam that has passed through the coating of the spherical optical structure and direct light is equivalent to half of the wavelength λ (half-wavelength), cases where this is equivalent to odd multiples of half-wavelengths are also included.

According to the present disclosure, the thickness D of the coating decreases as the wavelength λ of the structural color becomes shorter (bluer) and the difference in refractive index between the refractive index n₁ of the external medium and the refractive index of the coating substance n₂ increases, whereas the thickness D of the coating increases as the wavelength λ of the structural color becomes longer (redder), and the difference in refractive index between the refractive index n₁ of the external medium and the refractive index of the coating substance n₂ decreases. If all combinations of the external medium and the coating substance are considered, in at least one embodiment, the coating thickness D may range from 50 to 700 nm. For example, in cases where the external medium is a medium used in cosmetics such as water, alcohol, air, and the like, and the coating substance is a high molecular polymer, the coating thickness D may range from 80 to 500 nm, for example, from 100 to 300 nm.

Since the particle radius L has a small effect on the wavelength λ of the structural color in comparison with the coating thickness D, there are no restrictions on the particle diameter that are dependent on the wavelength of the structural color to be developed. However, in the case of a cosmetic coloring material or a cosmetic composition comprising these spherical optical structures, certain properties, for example, lustrous sensation, the depth of wrinkles in the skin, and the like, may be taken into consideration, and the particle diameter may be less than or equal to 500 μm. Furthermore, if the production of the structures and the control conditions are also taken into consideration, the particle diameter may range from 1 to 500 μm, for example, from 10 to 300 μm.

The spherical optical structures of the present disclosure may be produced according to methods used for the production of conventional microcapsules, for example, the interfacial polymerization method, the phase separation method, the interfacial precipitation method, the spray drying method, and the fluid bed method. However, the method of preparation is not restricted to conventional methods, and any method may be used. In at least one embodiment, the method may be suited to controlling the particle diameter and/or coating thickness of the spherical optical structures to be within a desired range.

In another embodiment, for the spherical optical structure of the present disclosure, ease of manipulation may be improved using the interfacial precipitation method.

The interfacial precipitation method is a microencapsulation method that is also called the secondary emulsion method, the submerged concentration method, the submerged drying method, and the like (see, for example, Microcapsules: Their Production, Properties, and Applications, Tamotsu Kondou et al., Sankyou Publishing). Interfacial precipitation may be classified into an interfacial precipitation method in water using a water/oil/water type compound emulsion, and an interfacial precipitation method in oil using an oil/water/oil type compound emulsion. It is understood that a skilled artisan will choose the method appropriately according to the properties of the materials to be utilized as the core substance and the coating substance.

For instance, if an aqueous solution of gelatin is used as the core substance, and a hydrophobic organic polymer such as polystyrene is used as the coating substance, the interfacial precipitation method in water may be used, which is explained in more detail below.

An oil/water emulsion (primary emulsion) is formed in which an about 4 to 8 wt % gelatin aqueous solution is dispersed in an about 1 to 5 wt % methylene chloride solution of polystyrene. Next, said primary emulsion is dispersed in water to form a water/oil/water type secondary emulsion. Treatments such as heating, pressure reduction, solvent extraction, freezing, cooling, and/or dry powder treatment may be optionally performed to obtain the spherical optical structures.

The solvent for dissolving the coating substance (hydrophobic polymer) in the interfacial precipitation method may be chosen as appropriate according to the type of polymer. Examples of suitable solvents include, but are not limited to, methylene chloride (for example, in the case of polystyrene and/or polycarbonate), benzene (for example, in the case of ethylcellulose), cyclohexane (for example, in the case of ethylcellulose), and carbon tetrachloride (for example, in the case of polystyrene).

According to the present disclosure, by adjusting the concentration of coating substance when preparing the primary emulsion, the coating thickness may be adjusted, and a spherical optical structure in the form of a microcapsule, that displays a desired structural color, may be obtained.

For example, if an about 5 wt % polystyrene solution (methylene chloride) is used, a spherical optical structure having a thickness ranging from 100 to 1000 nm may be obtained, and in a secondary emulsion wherein such spherical optical structures are dispersed in water, spherical optical structures developing a plurality of structural colors from red to blue may exist together.

On the other hand, if an about 1 wt % polystyrene solution is used, the ratio of spherical optical structures displaying a blue structural color may increase.

Additionally, the created secondary emulsion may contain particles of various sizes (particle diameters), but the spherical optical structures displaying structural colors may have particle diameters ranging from about 1 to 200 μm. However, there may be little or no correlation between the particle diameter of the spherical optical structures and the structural colors they display.

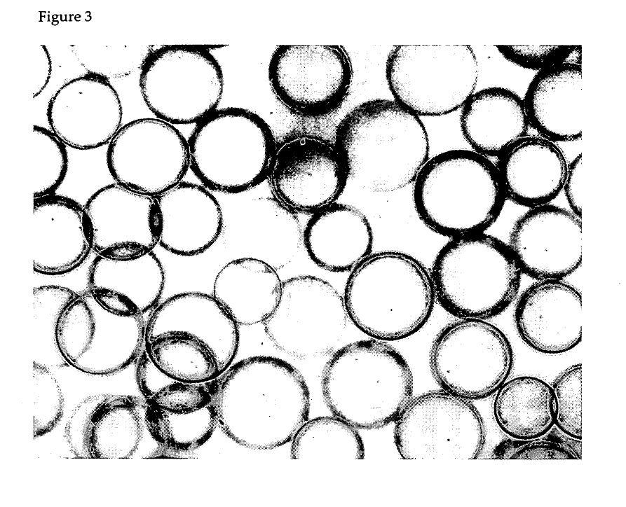

Although not wishing to be bound by theory, for the spherical optical structures of the present disclosure, it is believed that the greatest factor determining the structural colors thereof is the coating thickness. Therefore, as shown in FIG. 2, for example, if the other adjustable conditions are identical, the number of spherical optical structures displaying cold structural colors closer to blue may be increased by decreasing the coating substance concentration in the solution, whereas the ratio of spherical optical structures displaying warm structural colors closer to red may be increased if the coating substance concentration in the solution is not decreased.

Further, the particle diameter and/or coating thickness of the spherical optical structures may be controlled to be within a desired range by, for example, sifting using a differentiation means such as filtering and/or centrifuging on the rough structures produced by a conventional microcapsule preparation method and the like such as the interfacial precipitation method.

Spherical optical structures obtained by the methods discussed above may optionally be dried by performing a conventional treatment during the interfacial precipitation method, and used as solid particles, or they may be used as dispersants within a medium obtained as a secondary emulsion.

The spherical optical structure of the present disclosure may display hues having a unique transparent sensation. By mixing this spherical optical structure into cosmetic compositions as a cosmetic coloring material, a unique chromatic effect not obtainable conventionally may be obtained.

Therefore, disclosed herein is a cosmetic coloring material comprising at least one spherical optical structure displaying structural colors. Also disclosed herein is a cosmetic composition comprising said coloring material.

The cosmetic coloring material of the present disclosure may be in the form of dry particles or particles dispersed throughout a medium. Additionally, the cosmetic composition of the present disclosure may comprise, in addition to the spherical optical structures displaying structural colors, additives conventionally used in cosmetics, for example, anti-oxidants, fragrances, essential oils, preservatives, cosmetic activators, vitamins, necessary fatty acids, sphingolipids, self tanning compounds such as DHA, and sunscreen agents.

The cosmetic composition of the present disclosure may be applied to the skin of the face and body, mucous membranes, and/or keratin fibers such as nails, eyelashes, and/or hair.

These compositions may be in any form, for example, solid or flexible oil gels that may optionally comprise water; oil in water and water in oil multiple emulsions that are solid or gelatinized; and multi-phase systems, for example, two-phase systems. These compositions may have any outward appearance, for instance, creams, salves, soft pastes, ointments, and cast or molded solids, such as sticks. In at least one embodiment, the cosmetic composition may be in the form of a stick or a dish, for example, anhydrous hard gels and translucent or a transparent anhydrous sticks.

These compositions may be used as body health compositions, for example, deodorant sticks; hair compositions such as styling sticks and/or makeup sticks for the hair; makeup compositions for skin and/or mucous membranes of the face and/or the body, for example, lipsticks, foundations cast as a stick or a dish, face powders, eyeshadow, bases for coating onto conventional lipstick, concealer sticks, lip glosses, eyeliners, mascaras, and temporary tattoos; compositions for the care of skin and/or mucous membranes, for example, lip care balms or bases, body ointments, and daily care creams; and sunscreen compositions or self tanning compositions, and skincare compositions, for example, creams and facial cleansing gels.

The at least one spherical optical structure may be present in the cosmetic composition in an amount ranging from 0.001% to 74% by weight, for example, from 0.1% to 60% by weight, or from 1% to 30% by weight, relative to the total weight of the composition.

The cosmetic composition may additionally comprise additives such as oils, waxes, pigments, fillers, dyes, surfactants, water, solvents, alcohols, and mixtures thereof.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. In the examples that follow, the amounts of ingredients are expressed as percentages by weight with respect to the total weight of the composition, unless otherwise indicated.

### EXAMPLE

50 ml of 4 to 8 wt % aqueous gelatin solution was emulsified in 200 ml of methylene chloride solution containing 5 wt % of polystyrene (molecular weight 200,000), to prepare a water in oil type primary emulsion (1).

Separately from the above, 1500 ml of a 1 wt % aqueous gelatin solution was prepared, and the emulsion (1) was added under agitation, to obtain a water/oil/water type secondary emulsion.

By maintaining the temperature of the system at about 40° C., and evaporating the methylene chloride, spherical optical structures having a polystyrene shell and an aqueous solution as the core substance were obtained.

An optical photomicrograph of the obtained spherical optical structures is shown in FIG. 3. Each spherical optical structure displayed a structural color according to the thickness of its shell.

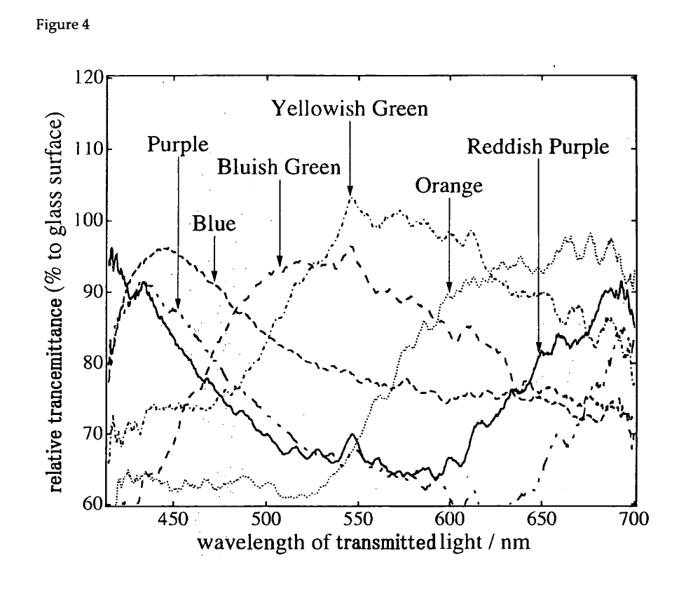

The transmission spectrum of spherical optical structures of each color obtained is shown in FIG. 4. It was confirmed that light transmitted by each of the spherical optical structures had a wavelength peak of transmitted light corresponding to each color.

What is claimed is:

1. A spherical optical structure having a particle diameter less than or equal to 500 μm, comprising a core substance and a coating substance coating the core substance, wherein the particle diameter and/or the thickness of the coating substance of said spherical optical structure are controlled to be in a predetermined range, according to the refractive index of an external medium in contact with the outer surface of said spherical optical structure and the refractive index of said coating substance, so as to display a structural color in the visible light range.

2. The spherical optical structure of claim 1, wherein the particle radius L and/or the thickness of the coating substance D of said spherical optical structure are controlled, according to the refractive index n₁ of the external medium in contact with the outer surface of said spherical optical structure and the refractive index n₂ of said coating substance, to display a structural color having a wavelength λ in the visible light range obtained by substituting a value d satisfying:
$$\begin{matrix}sin \{ 90 - arccos ( \frac{L - d}{L} ) \} = \frac{{n}_{2}}{{n}_{1}} sin [ \{ 90 - arccos ( \frac{L - d}{L} ) \} - arctan [ \frac{D - d}{L sin \{ arccos ( \frac{L - d}{L} ) \}} ] ] \\ into & Equation 1 \\ \frac{λ}{2} ( 2 m + 1 ) ( m = 0 , 1 , 2 , 3 , … ) = \frac{2 L sin \{ arccos ( \frac{L - d}{L} ) \}}{cos [ arctan [ \frac{D - d}{L sin \{ arccos ( \frac{L - d}{L} ) \}} ] ]} - 2 L sin \{ arccos ( \frac{L - d}{L} ) \} & Equation 2\end{matrix}$$
wherein m is an integer greater than or equal to 0.

3. The spherical optical structure of claim 1, wherein the thickness of the coating substance ranges from 50 to 700 nm.

4. The spherical optical structure of claim 1, wherein the thickness of the coating substance ranges from 80 to 500 nm.

5. The spherical optical structure of claim 1, wherein the thickness of the coating substance ranges from 100 to 300 nm.

6. The spherical optical structure of claim 1, having a particle diameter ranging from 1 to 500 μm.

7. The spherical optical structure of claim 1, having a particle diameter ranging from 10 to 300 μm.

8. The spherical optical structure of claim 1, wherein the core substance comprises a medium chosen from liquid and gas mediums.

9. The spherical optical structure of claim 8, wherein the liquid medium is chosen from water, hydro-organic solutions comprising water and/or at least one C₁-C₆ alcohol, aqueous liquid substances, organic liquid substances, inorganic solvents, and mixtures thereof.

10. The spherical optical structure of claim 9, wherein the at least one C₁-C₆ alcohol is chosen from ethanol and/or isopropanol.

11. The spherical optical structure of claim 8, wherein the liquid medium is chosen from water, hydro-organic solutions comprising water and/or at least one C₁-C₆ alcohol, gelatin or hydrophilic thickening agents, and mixtures thereof.

12. The spherical optical structure of claim 8, wherein the gas medium is air.

13. The spherical optical structure of claim 1, wherein the coating substance is chosen from vinyl based polymers, polycondensation polymers, cellulose based polymers, silicone based polymers, organic polymers, and/or inorganic materials.

14. The spherical optical structure of claim 13, wherein the coating substance is chosen from polystyrenes, polyethylenes, C₁-C₃₂ alkyl poly(meth)acrylates, methyl poly(meth)acrylates, and nylons.

15. A cosmetic composition comprising at least one liquid medium and at least one spherical optical structure, wherein
the at least one spherical optical structure is dispersed in the at least one liquid medium, and
the at least one spherical optical structure has a particle diameter less than or equal to 500 μm, comprising a core substance and a coating substance coating the core substance, wherein the particle diameter and the thickness of the coating substance of said spherical optical structure are controlled to be in a predetermined range, according to the refractive index of an external medium in contact with the outer surface of said spherical optical structure and the refractive index of said coating substance, so as to display a structural color in the visible light range.

16. A cosmetic coloring material comprising a spherical optical structure having a particle diameter less than or equal to 500 μm, comprising a core substance and a coating substance coating the core substance, wherein the particle diameter and the thickness of the coating substance of said spherical optical structure are controlled to be in a predetermined range, according to the refractive index of an external medium in contact with the outer surface of said spherical optical structure and the refractive index of said coating substance, so as to display a structural color in the visible light range.
